(19) United States
(12) Patent Application Publication
(10) Pub. No.: US 2024/0140817 A1
(43) Pub. Date: May 2, 2024

(54) COBALTOSIC OXIDE-LOADED TITANIUM DIOXIDE HETEROJUNCTION NANOZYME AND PREPARATION METHOD THEREFOR AND APPLICATION THEREOF

(71) Applicant: Shanghai Sixth People's Hospital, Shanghai (CN)

(72) Inventors: Longxiang SHEN, Shanghai (CN); Qingcheng YANG, Shanghai (CN); Bijiang GENG, Shanghai (CN); Wenyan SHI, Shanghai (CN); Dengyu PAN, Shanghai (CN)

(73) Assignee: Shanghai Sixth People's Hospital, Shanghai (CN)

(21) Appl. No.: 18/218,117

(22) Filed: Jul. 5, 2023

(30) Foreign Application Priority Data
Oct. 26, 2022 (CN) 202211315197.9

(51) Int. Cl. C01G 23/08; A61P 35/00; B82Y 5/00; B82Y 30/00; C01G 51/04

(52) U.S. Cl. CPC C01G 23/08; A61P 35/00; B82Y 5/00; B82Y 30/00; C01G 51/04; C01P 2002/74; C01P 2004/24

(57) ABSTRACT

A cobaltosic oxide-loaded titanium dioxide heterojunction nanozyme and a preparation method therefor and an application thereof are provided. The cobaltosic oxide-loaded titanium dioxide heterojunction nanozyme includes a surface oxygen vacancy doped TiO₂₋ₓ nanosheet and a variable-valence metal-containing Co₃O₄ nanozyme, wherein the Co₃O₄ nanozyme is loaded on the surface of the TiO₂₋ₓ nanosheet to construct and obtain the Co₃O₄@TiO₂₋ₓ heterojunction nanozyme. The Co₃O₄@TiO₂₋ₓ heterojunction nanozyme is used as a nanozyme to catalyze and generate endogenous O₂ and consume GSH, so as to improve the tumor microenvironment (TME), and achieve the effect of eradicating tumors by cooperating with sonodynamic therapy and catalytic therapy. The present Co₃O₄@TiO₂₋ₓ heterojunction nanozyme not only has an enhanced sonodynamic performance and tumor catalytic treatment performance, but also has tumor microenvironment regulation and control capabilities of relieving tumor hypoxia and consuming GSH, thus realizing the amplification of ROS quantum yield.

### CROSS REFERENCE TO THE RELATED APPLICATIONS

This application is based upon and claims priority to Chinese Patent Application No. 202211315197.9, filed on Oct. 26, 2022, the entire contents of which are incorporated herein by reference.

### TECHNICAL FIELD

The present invention relates to the fields of medicines and biotechnology, and in particular to a cobaltosic oxide-loaded titanium dioxide heterojunction nanozyme and a preparation method therefor and an application thereof.

### BACKGROUND

Malignant tumors are a class of refractory diseases that seriously threaten human life and health. At present, the conventional methods for clinically treating cancers, such as radiotherapy, chemotherapy, and surgery, still face problems such as large side effects, high recurrence rate, and limited therapeutic effect. This is due to the rapid proliferation of tumor cells and severe shortage of blood supply, TME has typical hypoxic characteristics, and the hypoxic area of the tumor is resistant to radiotherapy and chemotherapy due to lack of oxygen. In addition to the characteristics of severe hypoxia, the tumor microenvironment also has the problem of GSH overexpression. Overexpressed GSH will consume highly biotoxic reactive oxygen species (ROS), thereby reducing the efficacy of various tumor treatment modalities. In recent years, sonodynamic therapy, which stimulates sonosensitizers to generate ROS through the action of ultrasound, has been considered as an effective method to improve the status quo of cancer treatment. However, previously developed organic sonosensitizers can only rely on oxygen to generate singlet oxygen, and this single ROS generation mechanism is easily limited by the tumor hypoxic microenvironment. Compared with organic sonosensitizers, inorganic semiconductor nanomaterials such as titanium dioxide (TiO₂) have the advantages of chemical inertness to biological tissues, low cost, and easy fabrication, and therefore have been widely used in sonodynamic therapy (SDT) in recent years. However, the wide band gap structure of TiO₂ (3.2 eV) and the rapid recombination of electron-hole pairs (50±30 ns) decrease the quantum yield of ROS, resulting in poor SDT therapeutic effect. Therefore, designing and synthesizing a novel high-efficiency sonosensitizer, which has the capabilities of enhancing sonodynamic performance and regulating TME, is the focus and the difficulty of the current SDT research.

In view of this, the present invention provides a cobaltosic oxide-loaded titanium dioxide heterojunction nanozyme and a preparation method therefor and an application thereof.

### SUMMARY

Aiming at the deficiencies in the prior art, the present invention is intended to provide a cobaltosic oxide-loaded titanium dioxide heterojunction nanozyme, which aims to solve the problems of low ROS quantum yield, easy consumption of ROS by endogenous GSH, and the like of the existing nanosonosensitizer, and enhances the sonodynamic therapeutic effect of cancer by effectively regulating and controlling TME through catalyzing and generating endogenous O₂.

In order to solve the technical problems described above, the present invention provides the following technical solutions.

A cobaltosic oxide-loaded titanium dioxide heterojunction nanozyme comprises: a surface oxygen vacancy doped TiO₂₋ₓ nanosheet and a variable-valence metal-containing Co₃O₄ nanozyme, wherein the Co₃O₄ nanozyme is loaded on the surface of the TiO₂₋ₓ nanosheet to construct and obtain the Co₃O₄@TiO₂₋ₓ heterojunction nanozyme.

A preparation method for the cobaltosic oxide-loaded titanium dioxide heterojunction nanozyme comprises the following steps:
(a) 40-60 mg of TiO₂₋ₓ powders are weighed and added into 10-25 mL of ethanol, and the TiO₂₋ₓ powders are completely dissolved in the ethanol through ultrasonic treatment to obtain a TiO₂₋ₓ/ethanol mixed solution;
(b) the completely dissolved TiO₂₋ₓ/ethanol mixed solution is mixed with 20-60 mg of Co(CH₃COO)₂·4H₂O dissolved in 10-25 mL of ethanol, and a mixture is put into a reaction kettle for reaction, wherein a temperature in the reaction kettle is controlled to be 120-180° C. and a reaction time is controlled to be 3-5 h, to obtain a Co₃O₄@TiO₂₋ₓ heterojunction nanozyme primary mixed product; and
(c) after the Co₃O₄@TiO₂₋ₓ heterojunction nanozyme primary mixed product is cooled to room temperature, the primary mixed product is washed 2-5 times by using deionized water and ethanol, and is finally dried at a temperature of 40-80° C. to obtain Co₃O₄@TiO₂₋ₓ heterojunction nanozyme powders.

Further, a preparation method for the Co₃O₄ nanozyme comprises the following steps:

(1) 20-60 mg of Co(CH₃COO)₂·4H₂O is firstly weighed and dissolved in 10-25 mL of ethanol, and is put into a reaction kettle for reaction after complete dissolution, wherein a temperature in the reaction kettle is controlled to be 120-180° C. and a reaction time is controlled to be 3-5 h, to obtain a Co₃O₄ nanozyme reaction mixture;

(2) after the reaction is completed, the Co₃O₄ nanozyme reaction mixture is cooled to room temperature, and the Co₃O₄ nanozyme reaction mixture is taken out for centrifugation, and the reaction mixture is washed 2-5 times by using deionized water and ethanol, and is finally dried under vacuum at a temperature of 40-80° C. to obtain the Co₃O₄ nanozyme powders.

Further, a preparation method for the surface oxygen vacancy doped TiO₂₋ₓ nanosheet comprises the following steps:
(1) 0.5-2 g of white TiO₂ powders and 0.5-2 g of sodium borohydride are firstly weighed, put into a mortar, and ground in the same direction into uniform and fine powders, to obtain TiO₂/sodium borohydride mixture powders;
(2) the ground TiO₂/sodium borohydride mixture powders are then put into a horizontal tubular furnace for reaction under a nitrogen atmosphere, wherein a temperature in the horizontal tubular furnace is controlled to be 350-450° C. and a reaction time is controlled to be 2-5 h, to obtain black TiO₂/sodium borohydride mixture powders; and
(3) the obtained black TiO₂/sodium borohydride mixture powders are naturally cooled to room temperature, and the black TiO₂/sodium borohydride mixture powders are then slowly added into a 0.5-1.5 M hydrochloric acid solution and stirred for 1-2 h, and are centrifuged to collect and obtain a black precipitate, wherein the black precipitate is washed multiple times by using ethanol and deionized water and is finally dried under vacuum, to obtain the surface oxygen vacancy doped TiO₂₋ₓ nanosheet.

An application of a cobaltosic oxide-loaded titanium dioxide heterojunction nanozyme generating singlet oxygen in the sonodynamic performance, wherein the Co₃O₄@TiO₂₋ₓ heterojunction nanozyme prepared by the above method is enabled to generate a large amount of singlet oxygen under low-intensity ultrasound, and a singlet oxygen generation efficiency of the Co₃O₄@TiO₂₋ₓ heterojunction nanozyme under ultrasonic irradiation is detected by using 1,3-diphenylisobenzofuran as a singlet oxygen probe and detecting a change of absorption peak intensities at 412 nm under different ultrasonic times by using an ultraviolet spectrophotometer.

An application of a cobaltosic oxide-loaded titanium dioxide heterojunction nanozyme generating hydroxyl radicals in the chemical kinetic performance, wherein the Co₃O₄@TiO₂₋ₓ heterojunction nanozyme prepared by the above method is enabled to generate a large amount of hydroxyl radicals under low-intensity ultrasound, and a hydroxyl radical generation efficiency of the Co₃O₄@TiO₂₋ₓ heterojunction nanozyme under ultrasonic irradiation is detected by using 3,3,5,5-tetramethylbenzidine as a probe and detecting a change of absorption peak intensities at 654 nm under different ultrasonic times by using an ultraviolet spectrophotometer.

An application of a cobaltosic oxide-loaded titanium dioxide heterojunction nanozyme in consuming glutathione to improve the tumor microenvironment, wherein the Co₃O₄@TiO₂₋ₓ heterojunction nanozyme prepared by the above method is used to evaluate the Co₃O₄@TiO₂₋ₓ heterojunction nanozyme in consuming glutathione (GSH) to improve the tumor microenvironment by using 5,5′-dithiobis (2-nitrobenzoic acid) as a glutathione probe.

An application of a cobaltosic oxide-loaded titanium dioxide heterojunction nanozyme in promoting generation of endogenous O₂ to improve the tumor microenvironment, wherein the Co₃O₄@TiO₂₋ₓ heterojunction nanozyme prepared by the above method is used to detect oxygen generation amount of aqueous material solutions at different pH values after H₂O₂ at different concentrations is added by using a dissolved oxygen detector to improve the tumor microenvironment.

An application of a cobaltosic oxide-loaded titanium dioxide heterojunction nanozyme in in vivo sonodynamic reduction of tumor cells.

Use of a cobaltosic oxide-loaded titanium dioxide heterojunction nanozyme in preparing a medicine for treating tumors, wherein the medicine for treating tumors comprises a solvent, and 0.1-6 mg of a Co₃O₄@TiO₂₋ₓ heterojunction nanozyme is contained in every 1 mL of a medicine liquid, wherein the solvent and the Co₃O₄@TiO₂₋ₓ heterojunction nanozyme form a Co₃O₄@TiO₂₋ₓ tumor medicine liquid, and the Co₃O₄@TiO₂₋ₓ heterojunction nanozyme tumor medicine liquid is administrated to a receptor through intravenous injection.

Further, the solvent is normal saline.

Further, the receptor is an animal containing tumor cells.

With the adoption of the above technical solution, the present invention has the following beneficial effects:

The present invention provides a cobaltosic oxide-loaded titanium dioxide heterojunction nanozyme and a preparation method therefor and an application thereof. The Co₃O₄@TiO₂₋ₓ heterojunction nanozyme is used as a nanozyme to catalyze and generate endogenous O₂ and consume GSH, so as to improve the tumor microenvironment (TME), and achieve the effect of eradicating tumors by cooperating with sonodynamic therapy and catalytic therapy. The Co₃O₄@TiO₂₋ₓ heterojunction nanozyme prepared by the present invention not only has an enhanced sonodynamic performance and tumor catalytic treatment performance, but also has tumor microenvironment regulation and control capabilities of relieving tumor hypoxia and consuming GSH, thus realizing the amplification of ROS quantum yield. The nanozyme has a high biocompatibility, no obvious long-term toxicity, a low cost, a high activity, and a good thermal stability, and therefore is suitable for large-scale production.

### BRIEF DESCRIPTION OF THE DRAWINGS

The present invention is further illustrated below with reference to the accompanying drawing.

FIG. 1 is a TEM picture of Co₃O₄@TiO₂₋ₓ heterojunction nanozyme.

FIGS. 2A-2C are graphs of ¹O₂ generation rate comparison for Co₃O₄@TiO₂₋ₓ, and Co₃O₄; and

FIGS. 2D-2F are graphs of ·OH generation rate comparison for Co₃O₄@TiO₂₋ₓ, TiO₂₋ₓ, and Co₃O₄.

FIGS. 3A-3B are graphs of the performance test of Co₃O₄@TiO₂₋ₓ heterojunction nanozyme consuming GSH.

FIGS. 4A-4B are graphs comparing the generation rates of dissolved oxygen for Co₃O₄@TiO₂₋ₓ, TiO₂₋ₓ, and Co₃O₄ at the same pH and with the addition of the same concentration of H₂O₂.

FIGS. 5A-5C are graphs comparing cell viability of Co₃O₄@TiO₂₋ₓ heterojunction nanozyme under US irradiation (50 kHz, 2.0 Wcm⁻², 5 min) or without irradiation.

FIG. 6 is a graph of changes in tumor volume during tumor treatment.

### DETAILED DESCRIPTION OF THE EMBODIMENTS

In order to make the objectives, technical solutions and advantages of the present invention more apparent and clear, the present invention is further described in detail below with reference to the accompanying drawings and examples. It should be understood that the specific examples described herein are merely illustrative of the present invention and do not limit the scope of the present invention. Moreover, in the following description, descriptions of well-known structures and techniques are omitted to avoid unnecessarily obscuring the concept of the present invention.

### Example 1

Referring to FIG. 1, a cobaltosic oxide-loaded titanium dioxide heterojunction nanozyme comprises: a surface oxygen vacancy doped TiO₂₋ₓ nanosheet and a variable-valence metal-containing Co₃O₄ nanozyme, wherein the Co₃O₄ nanozyme is loaded on the surface of the TiO₂₋ₓ nanosheet to construct and obtain the Co₃O₄@TiO₂₋ₓ heterojunction nanozyme.

A preparation method for the cobaltosic oxide-loaded titanium dioxide heterojunction nanozyme comprises the following steps:
(a) 50 mg of TiO₂₋ₓ powders are weighed and added into 15 mL of ethanol, and the TiO₂₋ₓ powders are completely dissolved in the ethanol through ultrasonic treatment for 15 min to obtain a TiO₂₋ₓ/ethanol mixed solution;
(b) the completely dissolved TiO₂₋ₓ/ethanol mixed solution is mixed with 40 mg of Co(CH₃COO)₂·4H₂O dissolved in 15 mL of ethanol, and a mixture is put into a reaction kettle for reaction, wherein a temperature in the reaction kettle is controlled to be 150° C. and a reaction time is controlled to be 4 h, to obtain a Co₃O₄@TiO₂₋ₓ heterojunction nanozyme primary mixed product; and
(c) after the Co₃O₄@TiO₂₋ₓ heterojunction nanozyme primary mixed product is cooled to room temperature, the primary mixed product is washed 3 times by using deionized water and ethanol, and is finally dried at a temperature of 60° C. to obtain Co₃O₄@TiO₂₋ₓ heterojunction nanozyme powders.

Further, a preparation method for the Co₃O₄ nanozyme comprises the following steps:
(1) 40 mg of Co(CH₃COO)₂·4H₂O is firstly weighed and dissolved in 15 mL of ethanol, and is put into a reaction kettle for reaction after complete dissolution, wherein a temperature in the reaction kettle is controlled to be 150° C. and a reaction time is controlled to be 4 h, to obtain a Co₃O₄ nanozyme reaction mixture;
(2) after the reaction is completed, the Co₃O₄ nanozyme reaction mixture is cooled to room temperature, and the Co₃O₄ nanozyme reaction mixture is taken out for centrifugation, and the reaction mixture is washed 3 times by using deionized water and ethanol, and is finally dried under vacuum at a temperature of 60° C. to obtain the Co₃O₄ nanozyme powders.

Further, a preparation method for the surface oxygen vacancy doped TiO₂₋ₓ nanosheet comprises the following steps:
(1) 1 g of white TiO₂ powders and 1 g of sodium borohydride are firstly weighed, put into a mortar, and ground in the same direction into uniform and fine powders, to obtain TiO₂/sodium borohydride mixture powders;
(2) the ground TiO₂/sodium borohydride mixture powders are then put into a horizontal tubular furnace for reaction under a nitrogen atmosphere, wherein a temperature in the horizontal tubular furnace is controlled to be 400° C. and a reaction time is controlled to be 3 h, to obtain black TiO₂/sodium borohydride mixture powders; and
(3) the obtained black TiO₂/sodium borohydride mixture powders are naturally cooled to room temperature, and the black TiO₂/sodium borohydride mixture powders are then slowly added into a 1 M hydrochloric acid solution and stirred for 1-2 h, and are centrifuged to collect and obtain a black precipitate, wherein the black precipitate is washed 3 times by using ethanol and deionized water and is finally dried under vacuum, to obtain the surface oxygen vacancy doped TiO₂₋ₓ nanosheet.

Use of a cobaltosic oxide-loaded titanium dioxide heterojunction nanozyme in preparing a medicine for treating tumors, wherein the medicine for treating tumors comprises a solvent, and 0.1-6 mg of a Co₃O₄@TiO₂₋ₓ heterojunction nanozyme is contained in every 1 mL of a medicine liquid, wherein the solvent and the Co₃O₄@TiO₂₋ₓ heterojunction nanozyme form a Co₃O₄@TiO₂₋ₓ tumor medicine liquid, and the Co₃O₄@TiO₂₋ₓ heterojunction nanozyme tumor medicine liquid is administrated to a receptor through intravenous injection. The solvent is normal saline, and the receptor is an animal containing tumor cells.

### Example 2

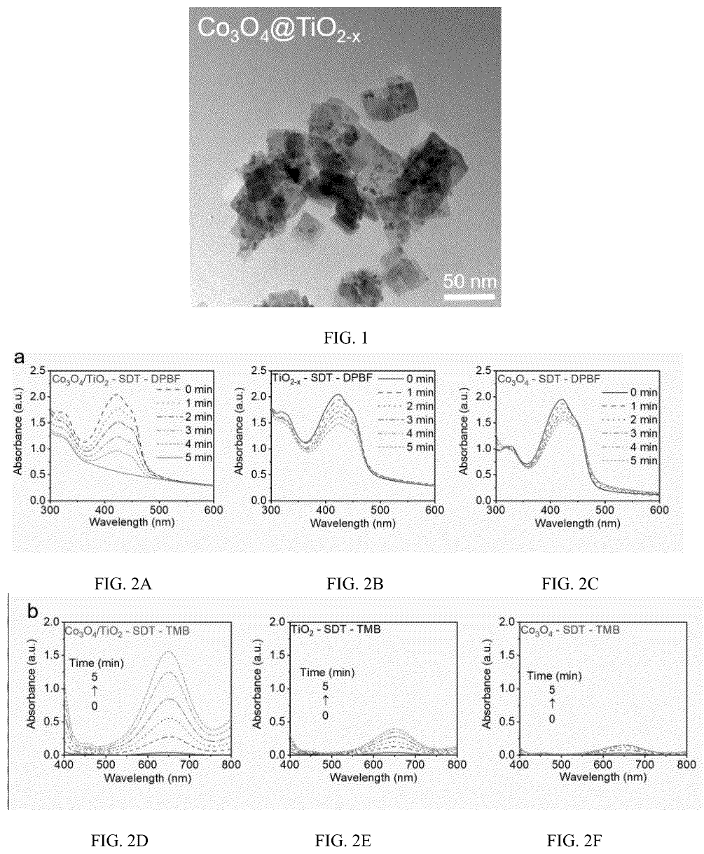

An application of a Co₃O₄@TiO₂₋ₓ heterojunction nanozyme generating singlet oxygen in the sonodynamic performance is characterized in that the Co₃O₄@TiO₂₋ₓ heterojunction nanozyme prepared by the above method is enabled to generate a large amount of singlet oxygen under low-intensity ultrasound, the chemical formula for singlet oxygen is ¹O₂, and a singlet oxygen generation efficiency of the Co₃O₄@TiO₂₋ₓ heterojunction nanozyme under ultrasonic irradiation is detected by using 1,3-diphenylisobenzofuran as a singlet oxygen probe and detecting a change of absorption peak intensities at 412 nm under different ultrasonic times by using an ultraviolet spectrophotometer, so as to evaluate the sonodynamic performance of the heterojunction nanozyme. Specifically, referring to FIGS. 2A-2C, the changes of the absorption peak intensity at 412 nm under different ultrasonic times such as 0 min, 1 min, 2 min, 3 min, 4 min, and 5 min are detected by an ultraviolet spectrophotometer, wherein FIG. 2A is the generation rate of ¹O₂ of Co₃O₄@TiO₂₋ₓ heterojunction nanozyme, FIG. 2B is the generation rate of ¹O₂ of TiO₂₋ₓ nanosheet, and FIG. 2C is the generation rate of ¹O₂ of Co₃O₄, and it can be seen that the generation rates of ¹O₂ of Co₃O₄@TiO₂₋ₓ heterojunction nanozyme are superior to that of TiO₂₋ₓ nanosheet and Co₃O₄.

An application of a Co₃O₄@TiO₂₋ₓ heterojunction nanozyme generating hydroxyl radicals in the chemical kinetic performance is characterized in that the Co₃O₄@TiO₂₋ₓ heterojunction nanozyme prepared by the above method is enabled to generate a large amount of hydroxyl radicals under low-intensity ultrasound, the chemical formula of hydroxyl radical is OH, and a hydroxyl radical generation efficiency of the Co₃O₄@TiO₂₋ₓ heterojunction nanozyme under ultrasonic irradiation is detected by using 3,3,5,5-tetramethylbenzidine as a probe and detecting a change of absorption peak intensities at 654 nm under different ultrasonic times by using an ultraviolet spectrophotometer, so as to evaluate the chemical kinetic performance of the heterojunction nanozyme. Specifically, referring to FIGS. 2D-2F, the changes of the absorption peak intensity at 654 nm under different ultrasonic times such as 0 min, 1 min, 2 min, 3 min, 4 min, and 5 min are detected by an ultraviolet spectrophotometer, wherein FIG. 2D is the generation rate of OH of Co₃O₄@TiO₂₋ₓ heterojunction nanozyme, FIG. 2E is the generation rate of OH of TiO₂₋ₓ nanosheet, and FIG. 2F is the generation rate of OH of Co₃O₄, and it can be seen that the generation rates of OH of Co₃O₄@TiO₂₋ₓ heterojunction nanozyme are superior to that of TiO₂₋ₓ nanosheet and Co₃O₄.

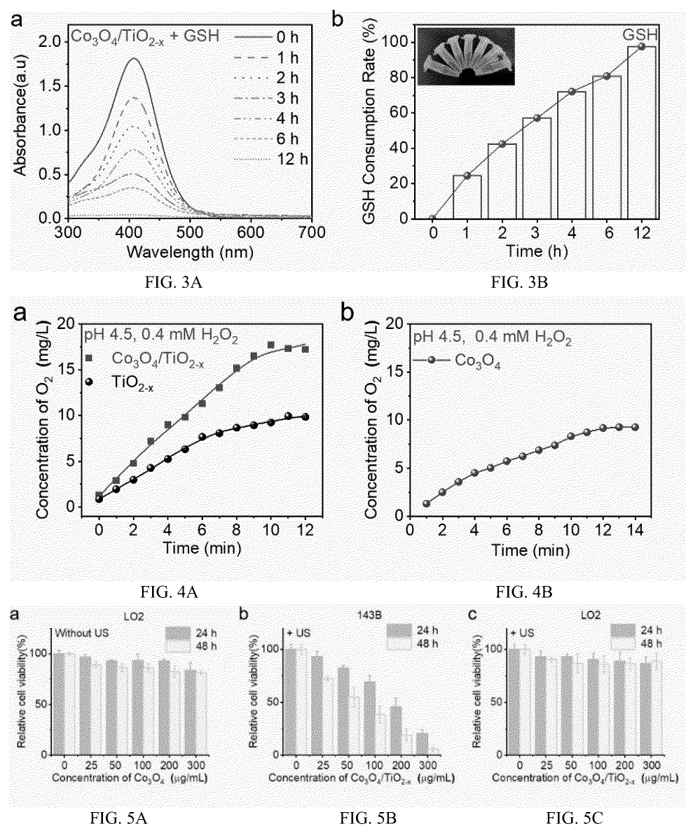

An application of a Co₃O₄@TiO₂₋ₓ heterojunction nanozyme in consuming glutathione (GSH) to improve the tumor microenvironment is characterized in that the Co₃O₄@TiO₂₋ₓ heterojunction nanozyme prepared by the above method is used to evaluate the Co₃O₄@TiO₂₋ₓ heterojunction nanozyme in consuming GSH to improve the tumor microenvironment by using 5,5′-dithiobis (2-nitrobenzoic acid) as a glutathione probe. It can be seen from FIGS. 3A-3B the amount of glutathione (GSH) consumed at different times such as 0 h, 1 h, 2 h, 3 h, 4 h, 6 h and 12 h by using 5,5′-dithiobis (2-nitrobenzoic acid) as a glutathione probe, wherein FIG. 3A is the changes of absorption peak intensity at different wavelengths and different times, and FIG. 3B is the amount of glutathione (GSH) consumed at different times by Co₃O₄@TiO₂₋ₓ heterojunction nanozyme, which indicates that the Co₃O₄@TiO₂₋ₓ heterojunction nanozyme can efficiently consume glutathione (GSH).

An application of a Co₃O₄@TiO₂₋ₓ heterojunction nanozyme in promoting generation of endogenous O₂ to improve the tumor microenvironment is characterized in that the Co₃O₄@TiO₂₋ₓ heterojunction nanozyme prepared by the above method is used to detect oxygen generation amount of aqueous material solutions at different pH values after H₂O₂ at different concentrations is added by using a dissolved oxygen detector to improve the tumor microenvironment. It can be seen from FIG. 4A and FIG. 4B that the catalytic ability of H₂O₂ of Co₃O₄@TiO₂₋ₓ heterojunction nanozyme, the catalytic ability of H₂O₂ of TiO₂₋ₓ nanosheet, and the catalytic ability of H₂O₂ of Co₃O₄ are compared at different times such as 0 min, 2 min, 4 min, 6 min, 8 min, 10 min, and 12 min, wherein FIG. 4A is the oxygen collection amount of H₂O₂ of Co₃O₄@TiO₂₋ₓ heterojunction nanozyme and TiO₂₋ₓ nanosheet, and FIG. 4B is the oxygen collection amount of Co₃O₄. This indicates that the catalytic ability of H₂O₂ of Co₃O₄@TiO₂₋ₓ heterojunction nanozyme is superior to that of TiO₂₋ₓ nanosheet and Co₃O₄.

### Example 3

An application of a Co₃O₄@TiO₂₋ₓ heterojunction nanozyme in in vivo sonodynamic reduction of tumor cells. Female nude mice aged 3-5 weeks were subcutaneously implanted with 100 μL (5 million) of human osteosarcoma cells (143B) at the armpit, and when the tumor volume reached 100 mm³, the nude mice were divided into 5 groups (5 per group).

Group (1), blank control group: specifically, 5 nude mice of group (1) were injected with 500 mL of normal saline every day without US irradiation.

Group (2), with US irradiation alone (50 kHz, 2.0 Wcm⁻², 5 min): specifically, 5 nude mice of group (2) were injected with 500 mL of normal saline every day and subjected to US irradiation (50 kHz, 2.0 Wcm⁻²) for 5 min.

Group (3), with Co₃O₄@TiO₂₋ₓ heterojunction nanozyme: specifically, 5 nude mice in the group (3) were injected with 500 mL of Co₃O₄@TiO₂₋ₓ heterojunction nanozyme normal saline solution every day, and each 1 mL of Co₃O₄@TiO₂₋ₓ heterojunction nanozyme normal saline solution contained 3 mg of Co₃O₄@TiO₂₋ₓ heterojunction nanozyme, and US irradiation was not performed.

Group (4), irradiated with Co₃O₄+US irradiation (50 kHz, 2.0 Wcm⁻², 5 min): specifically, 5 nude mice of group (4) were injected with 500 mL of Co₃O₄ normal saline solution every day, each 1 mL of Co₃O₄ normal saline solution contained 3 mg of Co₃O₄, and US irradiation (50 kHz, 2.0 Wcm⁻²) was performed for 5 min.

Group (5), with Co₃O₄@TiO₂₋ₓ+US irradiation (50 kHz, 2.0 Wcm⁻², 5 min): specifically, 500 mL of Co₃O₄@TiO₂₋ₓ heterojunction nanozyme normal saline solution was injected into 5 nude mice in the group (5) every day, and each 1 mL of Co₃O₄@TiO₂₋ₓ heterojunction nanozyme normal saline solution contained 3 mg of Co₃O₄@TiO₂₋ₓ heterojunction nanozyme, and US irradiation (50 kHz, 2.0 Wcm⁻²) was performed for 5 min.

Body weights of 25 nude mice were recorded every day and tumor volume size was measured every other day to evaluate the in vivo sonodynamic therapeutic efficacy of Co₃O₄@TiO₂₋ₓ heterojunction nanozyme.

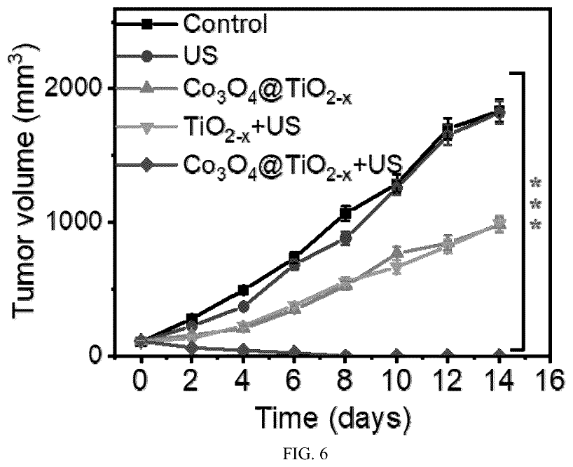

The experimental results are as follows with reference to FIGS. 5A-5C and FIG. 6:

The nude mice of group (1) had a tumor volume that increased from 100 mm 3 to 1855 mm³ after 14 days.

The nude mice of group (2) had a tumor volume that increased from 100 mm 3 to 1840 mm³ after 14 days.

The results of the group (3) are shown in FIG. 5A, the normal cell activity of the nude mice was not significantly changed, and it can be seen from FIG. 6 that the tumor volume of the nude mice increased from 100 mm 3 to 985 mm³ after 14 days.

The results of the group (4) are shown in FIG. 5B, the normal cell activity of the nude mice decreased, and it can be seen from FIG. 6 that the tumor volume of the nude mice increased from 100 mm 3 to 975 mm³ after 14 days.

The results of the group (5) are shown in FIG. 5C, and the normal cell activity of the nude mice was not significantly changed, and it can be seen from FIG. 6 that the tumor volume of the nude mice decreased from 100 mm 3 to 0 mm³ after 14 days.

According to the above experiments, referring to FIGS. 5A-5C, Co₃O₄@TiO₂₋ₓ heterojunction nanozyme has excellent biocompatibility; under the US irradiation, the survival rate of cancer cells is greatly reduced under the action of Co₃O₄@TiO₂₋ₓ heterojunction nanozyme, however, the survival rate of normal cells is not significantly changed, which indicates that the Co₃O₄@TiO₂₋ₓ heterojunction nanozyme can specifically kill the cancer cells.

According to the above experiments, referring to FIG. 6, under the US irradiation, Co₃O₄@TiO₂₋ₓ heterojunction nanozyme has the best tumor treatment effect compared with TiO₂₋ₓ nanosheet and Co₃O₄, and can completely inhibit the growth of tumors.

The present invention firstly achieves the purpose of reducing the band gap of TiO₂ by preparing a surface oxygen vacancy doped TiO₂₋ₓ nanosheet, so that electrons of TiO₂ are easier to be excited under the action of US; and then loads Co₃O₄ nanozyme containing valence-variable metal (Co^{2+/3+}) on the surface of the TiO₂₋ₓ nanosheet to construct Co₃O₄@TiO₂₋ₓ heterojunction nanozyme, so that the carrier transmission rate is greatly accelerated, the recombination of electron-hole pairs is inhibited, and the quantum yield of ROS is further improved. The Co₃O₄@TiO₂₋ₓ heterojunction nanozyme not only has peroxidase activity, which can catalyze hydrogen peroxide to generate a large amount of OH, but also has excellent catalase activity, which can catalyze hydrogen peroxide to generate a large amount of oxygen, thereby improving the performance of sonodynamic therapy (SDT). The high-valence metal ions (Co³⁺) in the Co₃O₄@TiO₂₋ₓ heterojunction nanozyme can also consume GSH, and achieve the purpose of improving TME, thereby enhancing the curative effect of tumor treatment.

The heterojunction nanozyme prepared in this example is subjected to related experiments such as characterization, detection of sonodynamic performance, detection of GSH consumption ability, detection of hydrogen peroxide catalytic ability, detection of intracellular GSH content, determination of cytotoxicity by an MTT method, treatment of tumors in vivo by an intravenous injection mode, and the like through instrument, and specific results are summarized as follows:

It can be seen from FIG. 1 that the TEM of Co₃O₄@TiO₂₋ₓ, heterojunction nanozyme shows that Co₃O₄ can be uniformly distributed on the surface of TiO₂₋ₓ, nanosheet.

It can be seen from FIGS. 2A-2F that the generation rates of ¹O₂ and OH of Co₃O₄@TiO₂₋ₓ heterojunction nanozyme are superior to those of TiO₂₋ₓ, nanosheet and Co₃O₄.

It can be seen from FIGS. 3A-3B that Co₃O₄@TiO₂₋ₓ, heterojunction nanozyme can efficiently consume glutathione (GSH).

It can be seen from FIGS. 4A-4B that the catalytic ability of H₂O₂ of Co₃O₄@TiO₂₋ₓ heterojunction nanozyme is superior to that of TiO₂₋ₓ nanosheet and Co₃O₄.

It can be seen from FIGS. 5A-5C that Co₃O₄@TiO₂₋ₓ heterojunction nanozyme has excellent biocompatibility; under the US irradiation, the survival rate of cancer cells is greatly reduced under the action of Co₃O₄@TiO₂₋ₓ heterojunction nanozyme, however, the survival rate of normal cells is not significantly changed, which indicates that the Co₃O₄@TiO₂₋ₓ heterojunction nanozyme can specifically kill the cancer cells.

It can be seen from FIG. 6 that, under the US irradiation, Co₃O₄@TiO₂₋ₓ heterojunction nanozyme has the best tumor treatment effect compared with TiO₂₋ₓ nanosheet and Co₃O₄, and can completely inhibit the growth of tumors.

The above are only specific examples of the present invention, and the technical features of the present invention are not limited thereto. Any simple changes, equivalent replacements or modifications based on the present invention to solve basically the same technical problems and achieve basically the same technical effects are covered by the protection scope of the present invention.

What is claimed is:

1. A cobaltosic oxide-loaded titanium dioxide (Co₃O₄@TiO₂₋ₓ) heterojunction nanozyme, comprising: a surface oxygen vacancy doped TiO₂₋ₓ nanosheet and a variable-valence metal-containing Co₃O₄ nanozyme, wherein the variable-valence metal-containing Co₃O₄ nanozyme is loaded on a surface of the surface oxygen vacancy doped TiO₂₋ₓ nanosheet to construct and obtain the Co₃O₄@TiO₂₋ₓ heterojunction nanozyme.

2. A preparation method for the cobaltosic oxide-loaded titanium dioxide (Co₃O₄@TiO₂₋ₓ) heterojunction nanozyme according to claim 1, comprising the following steps:
(a) weighing 40-60 mg of TiO₂₋ₓ powders and adding the TiO₂₋ₓ powders into 10-25 mL of ethanol, and completely dissolving the TiO₂₋ₓ powders in the ethanol through an ultrasonic treatment to obtain a TiO₂₋ₓ/ethanol mixed solution;
(b) mixing the TiO₂₋ₓ/ethanol mixed solution with 20-60 mg of Co(CH₃COO)₂·4H₂O dissolved in 10-25 mL of ethanol to obtain a mixture, and putting the mixture into a first reaction kettle for a first reaction to obtain a Co₃O₄@TiO₂₋ₓ heterojunction nanozyme primary mixed product, wherein a temperature in the first reaction kettle is controlled to be 120-180° C. and a reaction time of the first reaction is controlled to be 3-5 h; and
(c) cooling the Co₃O₄@TiO₂₋ₓ heterojunction nanozyme primary mixed product to room temperature, washing a cooled Co₃O₄@TiO₂₋ₓ heterojunction nanozyme primary mixed product 2-5 times by using deionized water and ethanol, and drying a washed Co₃O₄@TiO₂₋ₓ heterojunction nanozyme primary mixed product at a temperature of 40-80° C. to obtain Co₃O₄@TiO₂₋ₓ heterojunction nanozyme powders.

3. The preparation method for the cobaltosic oxide-loaded titanium dioxide (Co₃O₄@TiO₂₋ₓ) heterojunction nanozyme according to claim 2, wherein a preparation method for the variable-valence metal-containing Co₃O₄ nanozyme comprises the following steps:
(1) weighing 20-60 mg of the Co(CH₃COO)₂·4H₂O and dissolving the Co(CH₃COO)₂·4H₂O in 10-25 mL of ethanol to obtain a resulting solution, and putting the resulting solution into a second reaction kettle for a second reaction after a complete dissolution to obtain a Co₃O₄ nanozyme reaction mixture, wherein a temperature in the second reaction kettle is controlled to be 120-180° C. and a reaction time of the second reaction is controlled to be 3-5 h;
(2) after the second reaction is completed, cooling the Co₃O₄ nanozyme reaction mixture to room temperature, and taking out a cooled Co₃O₄ nanozyme reaction mixture for a centrifugation, washing a centrifuged Co₃O₄ nanozyme reaction mixture 2-5 times by using deionized water and ethanol, and drying a washed Co₃O₄ nanozyme reaction mixture under vacuum at a temperature of 40-80° C. to obtain Co₃O₄ nanozyme powders.

4. The preparation method for the cobaltosic oxide-loaded titanium dioxide (Co₃O₄@TiO₂₋ₓ) heterojunction nanozyme according to claim 2, wherein a preparation method for the surface oxygen vacancy doped TiO₂₋ₓ nanosheet comprises the following steps:
(1) weighing 0.5-2 g of white TiO₂ powders and 0.5-2 g of sodium borohydride, put the white TiO₂ powders and the sodium borohydride into a mortar to obtain a resulting mixture, and grinding the resulting mixture in a same direction into uniform and fine powders to obtain TiO₂/sodium borohydride mixture powders;
(2) putting the TiO₂/sodium borohydride mixture powders into a horizontal tubular furnace for a second reaction under a nitrogen atmosphere to obtain black TiO₂/sodium borohydride mixture powders, wherein a temperature in the horizontal tubular furnace is controlled to be 350-450° C. and a reaction time of the second reaction is controlled to be 2-5 h; and
(3) naturally cooling the black TiO₂/sodium borohydride mixture powders to room temperature, slowly adding cooled black TiO₂/sodium borohydride mixture powders into a 0.5-1.5 M hydrochloric acid solution for stirring for 1-2 h, and centrifuging a resulting solution to collect and obtain a black precipitate, washing the black precipitate multiple times by using ethanol and deionized water, and drying a washed black precipitate under vacuum to obtain the surface oxygen vacancy doped TiO₂₋ₓ nanosheet.

5. A method of an application of a cobaltosic oxide-loaded titanium dioxide (Co₃O₄@TiO₂₋ₓ) heterojunction nanozyme generating a singlet oxygen in a sonodynamic performance, wherein the Co₃O₄@TiO₂₋ₓ heterojunction nanozyme prepared by the method according to claim 2 is enabled to generate a large amount of the singlet oxygen under a low-intensity ultrasound, and a singlet oxygen generation efficiency of the Co₃O₄@TiO₂₋ₓ heterojunction nanozyme under an ultrasonic irradiation is detected by using 1,3-diphenylisobenzofuran as a singlet oxygen probe and detecting a change of absorption peak intensities at 412 nm under different ultrasonic times by using an ultraviolet spectrophotometer.

6. A method of an application of a cobaltosic oxide-loaded titanium dioxide (Co₃O₄@TiO₂₋ₓ) heterojunction nanozyme generating hydroxyl radicals in a chemical kinetic performance, wherein the Co₃O₄@TiO₂₋ₓ heterojunction nanozyme prepared by the method according to claim 2 is enabled to generate a large amount of the hydroxyl radicals under a low-intensity ultrasound, and a hydroxyl radical generation efficiency of the Co₃O₄@TiO₂₋ₓ heterojunction nanozyme under an ultrasonic irradiation is detected by using 3,3,5,5-tetramethylbenzidine as a probe and detecting a change of absorption peak intensities at 654 nm under different ultrasonic times by using an ultraviolet spectrophotometer.

7. A method of an application of a cobaltosic oxide-loaded titanium dioxide (Co₃O₄@TiO₂₋ₓ) heterojunction nanozyme in consuming glutathione (GSH) to improve a tumor microenvironment, wherein the Co₃O₄@TiO₂₋ₓ heterojunction nanozyme prepared by the method according to claim 2 is used to evaluate the Co₃O₄@TiO₂₋ₓ heterojunction nanozyme in consuming the GSH to improve the tumor microenvironment by using 5,5′-dithiobis (2-nitrobenzoic acid) as a glutathione probe.

8. A method of an application of a cobaltosic oxide-loaded titanium dioxide (Co₃O₄@TiO₂₋ₓ) heterojunction nanozyme in promoting a generation of endogenous O₂ to improve a tumor microenvironment, wherein the Co₃O₄@TiO₂₋ₓ heterojunction nanozyme prepared by the method according to claim 2 is used to detect an oxygen generation amount of aqueous material solutions at different pH values after H₂O₂ at different concentrations is added by using a dissolved oxygen detector to improve the tumor microenvironment.

9. A method of an application of a cobaltosic oxide-loaded titanium dioxide (Co₃O₄@TiO₂₋ₓ) heterojunction nanozyme in an in vivo sonodynamic reduction of tumor cells.

10. A method of a use of a cobaltosic oxide-loaded titanium dioxide (Co₃O₄@TiO₂₋ₓ) heterojunction nanozyme in preparing a medicine for treating tumors, wherein the medicine for treating the tumors comprises a solvent and 0.1-6 mg of the Co₃O₄@TiO₂₋ₓ heterojunction nanozymeper 1 mL of a medicine liquid, wherein the solvent and the Co₃O₄@TiO₂₋ₓ heterojunction nanozyme form a Co₃O₄@TiO₂₋ₓ tumor medicine liquid, and the Co₃O₄@TiO₂₋ₓ tumor medicine liquid is administrated to a receptor through an intravenous injection.

11. The method of the use of the cobaltosic oxide-loaded titanium dioxide (Co₃O₄@TiO₂₋ₓ) heterojunction nanozyme in preparing the medicine for treating the tumors according to claim 10, wherein the solvent is normal saline.

12. The method of the use of the cobaltosic oxide-loaded titanium dioxide (Co₃O₄@TiO₂₋ₓ) heterojunction nanozyme in preparing the medicine for treating the tumors according to claim 10, wherein the receptor is an animal containing tumor cells.

13. The method of the application of the cobaltosic oxide-loaded titanium dioxide (Co₃O₄@TiO₂₋ₓ) heterojunction nanozyme generating the singlet oxygen in the sonodynamic performance according to claim 5, wherein a preparation method for the variable-valence metal-containing Co₃O₄ nanozyme comprises the following steps:
(1) weighing 20-60 mg of the Co(CH₃COO)₂·4H₂O and dissolving the Co(CH₃COO)₂·4H₂O in 10-25 mL of ethanol to obtain a resulting solution, and putting the resulting solution into a second reaction kettle for a second reaction after a complete dissolution to obtain a Co₃O₄ nanozyme reaction mixture, wherein a temperature in the second reaction kettle is controlled to be 120-180° C. and a reaction time of the second reaction is controlled to be 3-5 h;
(2) after the second reaction is completed, cooling the Co₃O₄ nanozyme reaction mixture to room temperature, and taking out a cooled Co₃O₄ nanozyme reaction mixture for a centrifugation, washing a centrifuged Co₃O₄ nanozyme reaction mixture 2-5 times by using deionized water and ethanol, and drying a washed Co₃O₄ nanozyme reaction mixture under vacuum at a temperature of 40-80° C. to obtain Co₃O₄ nanozyme powders.

14. The method of the application of the cobaltosic oxide-loaded titanium dioxide (Co₃O₄@TiO₂₋ₓ) heterojunction nanozyme generating the singlet oxygen in the sonodynamic performance according to claim 5, wherein a preparation method for the surface oxygen vacancy doped TiO₂₋ₓ nanosheet comprises the following steps:
(1) weighing 0.5-2 g of white TiO₂ powders and 0.5-2 g of sodium borohydride, put the white TiO₂ powders and the sodium borohydride into a mortar to obtain a resulting mixture, and grinding the resulting mixture in a same direction into uniform and fine powders to obtain TiO₂/sodium borohydride mixture powders;
(2) putting the TiO₂/sodium borohydride mixture powders into a horizontal tubular furnace for a second reaction under a nitrogen atmosphere to obtain black TiO₂/sodium borohydride mixture powders, wherein a temperature in the horizontal tubular furnace is controlled to be 350-450° C. and a reaction time of the second reaction is controlled to be 2-5 h; and
(3) naturally cooling the black TiO₂/sodium borohydride mixture powders to room temperature, slowly adding cooled black TiO₂/sodium borohydride mixture powders into a 0.5-1.5 M hydrochloric acid solution for stirring for 1-2 h, and centrifuging a resulting solution to collect and obtain a black precipitate, washing the black precipitate multiple times by using ethanol and deionized water, and drying a washed black precipitate under vacuum to obtain the surface oxygen vacancy doped TiO₂₋ₓ nanosheet.

15. The method of the application of the cobaltosic oxide-loaded titanium dioxide (Co₃O₄@TiO₂₋ₓ) heterojunction nanozyme generating the hydroxyl radicals in the chemical kinetic performance according to claim 6, wherein a preparation method for the variable-valence metal-containing Co₃O₄ nanozyme comprises the following steps:
(1) weighing 20-60 mg of the Co(CH₃COO)₂·4H₂O and dissolving the Co(CH₃COO)₂·4H₂O in 10-25 mL of ethanol to obtain a resulting solution, and putting the resulting solution into a second reaction kettle for a second reaction after a complete dissolution to obtain a Co₃O₄ nanozyme reaction mixture, wherein a temperature in the second reaction kettle is controlled to be 120-180° C. and a reaction time of the second reaction is controlled to be 3-5 h;
(2) after the second reaction is completed, cooling the Co₃O₄ nanozyme reaction mixture to room temperature, and taking out a cooled Co₃O₄ nanozyme reaction mixture for a centrifugation, washing a centrifuged Co₃O₄ nanozyme reaction mixture 2-5 times by using deionized water and ethanol, and drying a washed Co₃O₄ nanozyme reaction mixture under vacuum at a temperature of 40-80° C. to obtain Co₃O₄ nanozyme powders.

16. The method of the application of the cobaltosic oxide-loaded titanium dioxide (Co₃O₄@TiO₂₋ₓ) heterojunction nanozyme generating the hydroxyl radicals in the chemical kinetic performance according to claim 6, wherein a preparation method for the surface oxygen vacancy doped TiO₂₋ₓ nanosheet comprises the following steps:
(1) weighing 0.5-2 g of white TiO₂ powders and 0.5-2 g of sodium borohydride, put the white TiO₂ powders and the sodium borohydride into a mortar to obtain a resulting mixture, and grinding the resulting mixture in a same direction into uniform and fine powders to obtain TiO₂/sodium borohydride mixture powders;
(2) putting the TiO₂/sodium borohydride mixture powders into a horizontal tubular furnace for a second reaction under a nitrogen atmosphere to obtain black TiO₂/sodium borohydride mixture powders, wherein a temperature in the horizontal tubular furnace is controlled to be 350-450° C. and a reaction time of the second reaction is controlled to be 2-5 h; and
(3) naturally cooling the black TiO₂/sodium borohydride mixture powders to room temperature, slowly adding cooled black TiO₂/sodium borohydride mixture powders into a 0.5-1.5 M hydrochloric acid solution for stirring for 1-2 h, and centrifuging a resulting solution to collect and obtain a black precipitate, washing the black precipitate multiple times by using ethanol and deionized water, and drying a washed black precipitate under vacuum to obtain the surface oxygen vacancy doped TiO₂₋ₓ nanosheet.

17. The method of the application of the cobaltosic oxide-loaded titanium dioxide (Co₃O₄@TiO₂₋ₓ) heterojunction nanozyme in consuming the glutathione (GSH) to improve the tumor microenvironment according to claim 7, wherein a preparation method for the variable-valence metal-containing Co₃O₄ nanozyme comprises the following steps:
(1) weighing 20-60 mg of the Co(CH₃COO)₂·4H₂O and dissolving the Co(CH₃COO)₂·4H₂O in 10-25 mL of ethanol to obtain a resulting solution, and putting the resulting solution into a second reaction kettle for a second reaction after a complete dissolution to obtain a Co₃O₄ nanozyme reaction mixture, wherein a temperature in the second reaction kettle is controlled to be 120-180° C. and a reaction time of the second reaction is controlled to be 3-5 h;
(2) after the second reaction is completed, cooling the Co₃O₄ nanozyme reaction mixture to room temperature, and taking out a cooled Co₃O₄ nanozyme reaction mixture for a centrifugation, washing a centrifuged Co₃O₄ nanozyme reaction mixture 2-5 times by using deionized water and ethanol, and drying a washed Co₃O₄ nanozyme reaction mixture under vacuum at a temperature of 40-80° C. to obtain Co₃O₄ nanozyme powders.

18. The method of the application of the cobaltosic oxide-loaded titanium dioxide (Co₃O₄@TiO₂₋ₓ) heterojunction nanozyme in consuming the glutathione (GSH) to improve the tumor microenvironment according to claim 7, wherein a preparation method for the surface oxygen vacancy doped TiO₂₋ₓ nanosheet comprises the following steps:
(1) weighing 0.5-2 g of white TiO₂ powders and 0.5-2 g of sodium borohydride, put the white TiO₂ powders and the sodium borohydride into a mortar to obtain a resulting mixture, and grinding the resulting mixture in a same direction into uniform and fine powders to obtain TiO₂/sodium borohydride mixture powders;
(2) putting the TiO₂/sodium borohydride mixture powders into a horizontal tubular furnace for a second reaction under a nitrogen atmosphere to obtain black TiO₂/sodium borohydride mixture powders, wherein a temperature in the horizontal tubular furnace is controlled to be 350-450° C. and a reaction time of the second reaction is controlled to be 2-5 h; and
(3) naturally cooling the black TiO₂/sodium borohydride mixture powders to room temperature, slowly adding cooled black TiO₂/sodium borohydride mixture powders into a 0.5-1.5 M hydrochloric acid solution for stirring for 1-2 h, and centrifuging a resulting solution to collect and obtain a black precipitate, washing the black precipitate multiple times by using ethanol and deionized water, and drying a washed black precipitate under vacuum to obtain the surface oxygen vacancy doped TiO₂₋ₓ nanosheet.

19. The method of the application of the cobaltosic oxide-loaded titanium dioxide (Co₃O₄@TiO₂₋ₓ) heterojunction nanozyme in promoting the generation of endogenous O₂ to improve the tumor microenvironment according to claim 8, wherein a preparation method for the variable-valence metal-containing Co₃O₄ nanozyme comprises the following steps:
(1) weighing 20-60 mg of the Co(CH₃COO)₂·4H₂O and dissolving the Co(CH₃COO)₂·4H₂O in 10-25 mL of ethanol to obtain a resulting solution, and putting the resulting solution into a second reaction kettle for a second reaction after a complete dissolution to obtain a Co₃O₄ nanozyme reaction mixture, wherein a temperature in the second reaction kettle is controlled to be 120-180° C. and a reaction time of the second reaction is controlled to be 3-5 h;
(2) after the second reaction is completed, cooling the Co₃O₄ nanozyme reaction mixture to room temperature, and taking out a cooled Co₃O₄ nanozyme reaction mixture for a centrifugation, washing a centrifuged Co₃O₄ nanozyme reaction mixture 2-5 times by using deionized water and ethanol, and drying a washed Co₃O₄ nanozyme reaction mixture under vacuum at a temperature of 40-80° C. to obtain Co₃O₄ nanozyme powders.

20. The method of the application of the cobaltosic oxide-loaded titanium dioxide (Co₃O₄@TiO₂₋ₓ) heterojunction nanozyme in promoting the generation of endogenous O₂ to improve the tumor microenvironment according to claim 8, wherein a preparation method for the surface oxygen vacancy doped TiO₂₋ₓ nanosheet comprises the following steps:
(1) weighing 0.5-2 g of white TiO₂ powders and 0.5-2 g of sodium borohydride, put the white TiO₂ powders and the sodium borohydride into a mortar to obtain a resulting mixture, and grinding the resulting mixture in a same direction into uniform and fine powders to obtain TiO₂/sodium borohydride mixture powders;
(2) putting the TiO₂/sodium borohydride mixture powders into a horizontal tubular furnace for a second reaction under a nitrogen atmosphere to obtain black TiO₂/sodium borohydride mixture powders, wherein a temperature in the horizontal tubular furnace is controlled to be 350-450° C. and a reaction time of the second reaction is controlled to be 2-5 h; and
(3) naturally cooling the black TiO₂/sodium borohydride mixture powders to room temperature, slowly adding cooled black TiO₂/sodium borohydride mixture powders into a 0.5-1.5 M hydrochloric acid solution for stirring for 1-2 h, and centrifuging a resulting solution to collect and obtain a black precipitate, washing the black precipitate multiple times by using ethanol and deionized water, and drying a washed black precipitate under vacuum to obtain the surface oxygen vacancy doped TiO₂₋ₓ nanosheet.
